(19) United States
(12) Patent Application Publication
(10) Pub. No.: US 2012/0100605 A1
(43) Pub. Date: Apr. 26, 2012

(54) DEVICE FOR CULTIVATING CELLS AND/OR MICROORGANISMS

(75) Inventors: Joerg Kauling, Bersch Gladbach (DE); Bernd Meuser, Koeln (DE); Arndt Braun, Remscheid (DE); Sebastien Chaussin, Aubagne (FR); Wilfgang Kahlert, Koerle (DE)

(73) Assignees: SARTORIUS STEDIM BIOTECH GMBH, GOETILINGEN (DE); BAYER TECHNOLOGY SERVICES GMBH, LEVERKUSEN (DE)

(21) Appl. No.: 13/318,660

(22) PCT Filed: Dec. 3, 2009
(86) PCT No.: PCT/EP09/08616

§ 371 Date: Jan. 10, 2012

(30) Foreign Application Priority Data
May 5, 2009 (DE) 10 2009 019 697.8

(51) Int. Cl. C12M 1/00; C12N 5/07

(52) U.S. Cl. 435/325; 435/289.1

(57) ABSTRACT

A reactor is designed as a disposable element, a container receives the reactor, and a device comprises a reactor. A drive unit of the device generates a rotating-oscillating motion of the reactor, and the device is utilized for cultivating cells and/or microorganisms.

The invention relates to a reactor which is preferably designed as a disposable element, a container for accommodating the reactor, a device comprising a reactor and a drive unit for generating a rotating-oscillating motion of the reactor, and the use of the device for cultivating cells and/or microorganisms.

In the highly regulated production of pharmaceuticals, a large expenditure in terms of time, equipment and personnel is apportioned to the provision of cleaned and sterilized bioreactors. In order reliably to avoid cross-contamination during a product change in a multipurpose plant or between two product batches, apart from the cleaning, a very complex cleaning validation is required which may need to be repeated in the event of a process adaptation.

This applies not only to upstream processing, USP, that is to say the production of biological products in fermenters, but also to downstream processing, DSP, that is to say purification of the fermentation products.

In USP and DSP, use is frequently made of kettles as agitator and reaction system. Especially in the case of fermentation, an aseptic environment is essential for successful culturing. For the sterilization of batch or fed-batch fermenters, the SIP (SIP=sterilizable-in-place) technique is generally used. In order in the case of continuous process procedure to ensure sufficient long-term sterility, use is also made of the autoclave technique, but the latter requires laborious transport of the reactors to the autoclave and is only usable in comparatively small reactor scales. The risk of contamination during fermentation is particularly critical during sampling and at moving stirrer shafts. The latter are generally equipped with complex sealing systems (e.g.: sliding-ring seals). Technologies which succeed without such penetration of the fermentation casing are preferred because of their greater process robustness.

The reactor downtime necessitated by the preparation procedures can be, in particular in the case of short use periods and frequent change of product, of the order of magnitude of reactor availability. The affected steps in the USP of biotechnological production are, for example, the process steps of media production and fermentation, and, in the DSP, solubilization, freezing, thawing, pH adjustment, precipitation, crystallization, buffer exchange and virus inactivation.

In order to meet the demand for a rapid and flexible recharging of the production plant while maintaining maximum cleanliness and sterility, designs for disposable reactors are the subject of constantly growing interest on the market.

WO2007/121958A1 describes a disposable reactor of this type for cultivating cells and microorganisms. In a preferred embodiment, said disposable reactor is composed of a stable, preferably multi-layered polymer material. The deformable disposable reactor is accommodated by a container which supports it. In this case, it is preferably introduced into the container from above. The container is connected to a drive unit. The drive unit sets the container, including the disposable reactor, into a rotating-oscillating motion about a positionally fixed, preferably vertical axis of the container. By means of an angular embodiment of the disposable reactor and/or of the internals in the disposable reactor, a high power input into the reactor contents can be achieved during the oscillating-rotating motion, and therefore the disposable reactor can be used as a fermenter with surface gas introduction for cultivating cells and microorganisms.

The disposable reactor described in WO2007/121958A1 has a flat floor or an outwardly curved pyramidal floor with a central outlet. The effect intended to be achieved by the outwardly curved pyramidal floor with the central outlet is that, once a valve in the outlet is opened, complete removal of the reactor contents is possible via a hose line.

A disadvantage of the system described in WO2007/121958A1 is that problems which play a secondary role at a reactor volume of 10 L to 100 L occur as the size of the disposable reactor increases.

A flat floor has the disadvantage, for example, that the disposable reactor cannot readily be completely emptied: if an outlet is above the floor edge, some of the reactor contents remains in the reactor. This may be considerable quantities of product in the case of a large-volume reactor. The outwardly curved floor with the central outlet affords the advantage here that the reactor can be virtually completely emptied.

However, as the size of the disposable reactor increases, it becomes increasingly difficult to introduce said disposable reactor from above into a container. Furthermore, it becomes increasingly more difficult to attach a hose line from the outside to the central outlet of the pyramidally outwardly curved floor of the disposable reactor. Moreover, a downwardly guided hose line constitutes a safety risk: in the event of nontight coupling or should the hose line burst, the entire reactor contents will run out without obstruction.

In addition, as the size of the reactor increases, the mixing action of the rotating-oscillating motion is reduced in the vicinity of the axis of rotation. There is the risk, when cultivating cells and/or microorganisms, that the cultures located in the vicinity of the axis of rotation will not obtain a sufficient supply of nutrients.

By means of the decreasing surface/volume ratio as the reactor size increases, there is the risk, in particular in the case of relatively large reactors, of cells and/or microorganisms being insufficiently supplied with gases if said gases are introduced into the reactor exclusively by surface gas introduction.

Starting from the prior art, it is the object to provide a system for carrying out processes with high demands imposed on cleanliness and sterility, the system reducing the expenditure in terms of time, equipment and personnel being apportioned to the provision of cleaned and sterilized components. The system is intended in particular to be usable even for process volumes of 100 L to 200 L and greater. It is intended to meet the excepting demands of the pharmaceutical industry, and to be handled simply and intuitively and to be cost-effective. It is intended to reduce safety risks due to the emergence of substances from the process space to a minimum. It is intended to permit sufficient mixing of the process contents. It is intended to be suitable for the cultivation of microorganisms and cell cultures and to ensure sufficient supply of the culture medium with in particular gaseous substances, and sufficient removal thereof from the culture medium.

According to the invention, this object is achieved by the subject matter cited in the independent claims. Preferred embodiments are found in the dependent claims.

A first subject matter of the present invention is a reactor comprising walls which enclose an interior space, and passages and/or connections in the walls, permitting connection of the interior space to the external surroundings, characterized in that the reactor, when filled with liquid, has a polygonal cross section parallel to the surface of the liquid and a floor which is curved or can be curved into the interior space.

The reactor according to the invention constitutes a space which can be sealed off from the outside, for carrying out chemical, biological, biochemical and/or physical processes. In particular, the reactor serves to provide a space for cultivating cells and/or microorganisms.

The reactor is preferably designed as a disposable element, i.e. it is preferably provided not to clean the reactor after use but rather to dispose of the reactor. The reactor therefore comprises only the essential elements required to provide a sterile reaction space: walls which enclose a space and seal off the latter from the outside, and passages in the walls, in order to be able to connect the reaction space to the outside world in a controlled manner.

All of the remaining elements which are required for operating a reactor, in particular for cultivating cells and/or microorganisms, are provided by peripheral equipment which is reusable. Said peripherel equipment, which comprises a container for accommodating the reactor and a drive unit for the rotating-oscillating movement of the reactor, is described in more detail further below.

The reactor, which conventionally constitutes a cohesive unit in the prior art, is therefore preferably divided in the present case into separate parts which are configured in accordance with the functions thereof In this case, the reactor and the container, as separate parts of an overall system, are matched to each other in such a manner that the reactor can be placed into the container and, when filled with liquid, is supported by said container. The reactor can be interpreted as the inner skin of the container, which can be renewed in order to ensure an unused, sterile reaction cell for a process change.

The reactor according to the invention is designed in such a manner that it can be produced cost-effectively. The reactor is preferably a deformable, hollow body, the walls of which are flexible, i.e. pliant, and the shape of which, at least in the unfilled state of the reactor, can be changed by an external force. The walls are preferably composed of a stable, single- or multi-layered polymer material. The deformability makes it possible, for example, to fold the reactor and therefore to bring the latter into a space-saving shape for transport and storage.

In the case of a reactor which is preferably designed as a disposable element, the deformability also arises from the requirement for low material and production costs for the reactor. Since the reactor is essentially intended to provide only a sterile space, it is not necessary, for example, for the walls to be self-supporting. Instead, the reactor is designed as a deformable body which, for use, is placed into a container which supports the reactor. The wall strength of the reactor can therefore be reduced to a minimum, which meets the stability criteria for a supported reactor. The wall strength thus customarily lies within a range in which the walls are deformable.

Furthermore, a deformable reactor with flexible walls can fit tightly against the container regions which are intended to support the reactor during the use thereof, which can be advantageous, for example, for compensating for manufacturing tolerances.

The walls of the reactor according to the invention (and therefore preferably also those container regions which serve to accommodate the reactor) are arranged in such a manner that power is input into the liquid contents of the reactor during a rotating-oscillating motion of the reactor about a positionally fixed axis.

If the reactor was designed as a circular cylinder and the reactor which is filled with liquid executed a rotating-oscillating motion about the longitudinal axis of the cylinder, the inert liquid in the interior of the reactor would scarcely move together with the reactor. Only the layer close to the wall would be forced to rotate at the same time due to the adhesion forces. Therefore, the reactor according to the invention, when filled with liquid, has an angular cross section parallel to the surface of the liquid. The angular embodiment ensures that power is input into the liquid and impresses the motion of the reactor on to the reactor contents. The reactor moves in an oscillating manner, i.e. the direction of rotation is periodically changed. Owing to the inertia and the fluid state of the reactor contents, the motion of the reactor contents lags behind the motion of the reactor. A mixing action is thereby obtained. In particular, it is thereby possible to input gas into the liquid above the liquid. This is explained by way of example on page 25 and in FIG. 5c of laid-open specification WO2007/121958A1.

The cross section parallel to the surface of the liquid of the filled reactor preferably has the shape of an n corner, where n is in the region of 3 to 12, preferably in the region of 4 to 8, very particularly preferably in the region of 4 to 6, and most preferably n is equal to 4.

The side walls of the reactor according to the invention are preferably at least partially designed as plane surfaces which do not run parallel to the surface of the liquid and meet one another at an angle of 45° to 120°.

It is conceivable, for example, that the side walls of the reactor form a polyhedron.

The reactor according to the invention furthermore has a floor which is fitted into the interior space of the reactor or, in the case of a deformable reactor, a floor which can be fitted inward. The floor may be, for example, in the shape of an inwardly directed tetrahedron, an inwardly directed pyramid, or have the shape of a paraboloid or of a bell. Further shapes are conceivable.

The floor preferably has inwardly directed edges which, during a rotating-oscillating motion, cause power to be input into the fluid located in the reactor. The floor is particularly preferably of pyramidal design.

The region which is fitted or can be fitted into the interior space is also referred to below as a curvature.

The height h of the curvature lies within the range of 0.01 times to 1 times the circle-equivalent diameter d of the floor cross section. If the floor cross section is, for example, square with a side length a, as may be the case in a pyramidally curved floor, the cross-sectional area is A=a². The circle-equivalent diameter d is then, according to formula 1:

$$d = \sqrt{\frac{4 A}{π}} \qquad ( 1 )$$

The height h of the curvature to the circle-equivalent diameter d is preferably within the range of 3% to 100%, particularly preferably within the range of 5% to 30% and very particularly preferably within the range of 10% to 20%.

It has surprisingly been found that an inwardly curved/fitted floor, in particular if it is of polyhedral (for example pyramidal) design and therefore has edges directed into the interior of the reactor, considerably improves the mixing action of the rotating-oscillating reactor in comparison to a floor of flat design or fitted outward. This can be attributed to a power input which is increased by the inwardly directed edges, during the rotating-oscillating movement. Furthermore, a floor shape with inwardly directed edges causes re-suspension of particles which drop to the floor in the interior of the reactor. The oscillating motion gives rise at the edges to vortices which convey the particles back into the upper region of the reactor again.

Furthermore, the inner curvature reduces the residual volume when removing the reactor contents via lateral connections above the floor edge of the reactor. This makes it possible to avoid drainage connections reaching into the floor and the associated problems and risks.

The reactor according to the invention has passages and/or connections in order, for example, to be able to remove and/or supply substances and/or to carry out measurements on the reactor contents.

Since the reactor is intended to execute a rotating-oscillating motion about a positionally fixed axis, the inclination is to bring passages and/or connections into the vicinity of the axis of rotation in order, for example, to keep hose lengths for the supply and/or removal of substances as short as possible. Hoses can be guided away from the reactor along the axis of rotation in a simple manner in this case and do not become tangled so rapidly. Also in the laid-open specification WO2007/121958A1, hoses are preferably mounted in the head region of the reactor in the vicinity of the axis of rotation.

However, it has turned out that connections are disadvantageous in the head region, in particular if hose ends are involved, which are intended to project from above into the reactor contents (dip tubes), since said hose ends, due to the rotating-oscillating movement, do not adopt a reproducible position in the reactor, but rather dangle to and fro. Hose ends of this type have to be placed into the reactor interior during the production of the reactor, which results in increased production costs. The hose ends can easily be tangled during the production of the reactor, during transport, during commissioning and during operation. They can only be unraveled with very great difficulty, if at all, from the outside, since the reactor interior is not accessible from the outside, in particular during operation.

The reactor according to the invention therefore preferably has passages and/or connections in the side region.

A passage for letting out the reactor contents is preferably mounted in the vicinity of the floor edge.

It is possible, for example, for the pH value and/or the oxygen concentration in the reactor to be measured via the connections which are likewise mounted laterally. Measuring systems which operate contactlessly, such as fluorescence sensors which can be interrogated optically (also see in this respect WO2007/121958A1, in which sensors of this type are described in more detail), are particularly suitable.

The reactor according to the invention is preferably formed from a single- or multi-layered transparent polymer material permitting a view into the reactor during operation.

Polymer material is a comparatively cost-effective material which can also be processed comparatively cost-effectively. The disposal of the used reactor and the use of a new disposable reactor are therefore more economical than cleaning used reactors, in particular since a complex cleaning validation is not needed for the use of a new disposable reactor. The reactor according to the invention is preferably packaged aseptically.

Suitable materials for the reactor according to the invention are in particular the materials and material combinations used in the patent U.S. Pat. No. 6,186,932B1, in columns 2 and 3 for the sachets mentioned there. The wall thicknesses cited there can also be transferred to reactors according to the invention.

In a preferred embodiment, the walls of the reactor according to the invention are composed of a film composite comprising a layer of polypropylene and a layer of polyethylene.

The polyethylene layer is preferably located on the inside of the reactor while the polypropylene layer is preferably located on the outside of the reactor.

The reactor according to the invention of polymer films can be produced, for example, according to the method described in U.S. Pat. No. 6,186,932B1, with the weld seams having to be adapted in such a manner that a floor which can be fitted inward is produced. An exemplary embodiment for the production of a preferred embodiment of a reactor according to the invention is described further below.

The reactor according to the invention has a ratio of height to the maximum width within the range of 0.2 to 3, preferably 0.5 to 2, particularly preferably 0.7 to 1.5.

The reactor volume can assume, for example, values of 10 L to 300 L.

For the correct use, the reactor is preferably placed into a container which supports the flexible walls of the reactor when the reactor is filled. The shapes of the container and reactor are therefore preferably coordinated with each other.

The container for accommodating a reactor according to the invention is a further subject matter of the present invention.

The container according to the invention comprises at least
an interior space for accommodating a reactor,
an opening for the frontal introduction of a reactor into the container, wherein the opening can be closed and is designed to be liquid-tight in the closed state,
a floor which is curved inward into the interior space of the container, and
a side channel via which hoses and/or channels and/or measuring probes can be led up to the reactor.

A container is understood as meaning a container which encloses an interior space and, in the closed state, delimits the interior space from the outside world by means of walls. The container according to the invention is preferably designed to be liquid-tight, i.e. it can be sealed from the outside world in such a manner that liquid does not inadvertently pass from the interior of the container into the outside world.

The container may be of cylindrical design, for example in the form of a column, or designed as a cube. The container is preferably of cuboidal design. The width and depth of the container are particularly preferably approximately equal in size with respect to the interior space without a side channel, i.e. they differ from each other by 200% at maximum. The ratio of height to the maximum width is 0.2 to 3, preferably 0.5 to 2 and particularly preferably 0.7 to 1.5. The sometimes comparatively small ratios of height to width permit the accommodating of large volumes in small spaces, for example in laboratories, and improve the substance transport during surface gas introduction as a consequence of the comparatively high specific surface of the liquid per reactor volume.

A reactor according to the invention can be placed into the interior space of the container. The preferably deformable reactor is supported on the inner walls of the container. The container has an inwardly curved floor. The floor shapes of the container and of the reactor are coordinated with each other.

In addition to the improved mixing action and the smaller dead space, the inward curvature additionally has the advantage, when the reactor contents are being let out, of increasing the stability of the container. As a result, the container wall thicknesses can be reduced and a weight and therefore cost saving achieved.

Depending on requirements regarding the pressure in the head space, the cover plate of the container can be designed in such a manner that forces can be transmitted from the reactor to the container. In this case, care should be taken to ensure a very substantial frictional connection between the reactor and container.

The container has at least one opening which is provided on one side of the container and via which the reactor can be placed, preferably frontally, into the container. In contrast to the system described in WO2007/121958A1, the reactor is preferably not placed into the container from above, but rather frontally. In the case of reactors having volumes of 100 L to 200 L and greater, the container reaches a height such that placing the reactor into position from above firstly requires a corresponding room height and secondly requires aids in order to reach an upper container opening (ladder, crane). Owing to the simpler handling, a frontally directed opening is advantageous.

The opening is of closable design, i.e. a door or the like is present in order to be able to close the container preferably in a liquid-tight manner.

The container furthermore has a side channel via which hoses and other connections, for example sensors or sensor lines, can be led up to the reactor. The channel is sealed off to the outside such that, in the event of leakage, no media, in particular no biological media with genetically modified organisms, can escape from the container.

In a preferred embodiment, the channel is formed by two spaced-apart, preferably transparent walls, which run into the floor region of the disposable reactor. The inner wall which is directed toward the reactor serves to support the reactor. The outer wall serves to seal off the container to the outside. Both walls can be removed for the frontal introduction of the reactor into the container. The hose lines and other couplings are guided through the channel upward and, above the reactor, to the axis of rotation of the container and are led out of the installation above the vertical axis. This therefore reliably avoids the reactor completely draining as a consequence of a leakage in a coupling. Additional protection of the lines from the surroundings is therefore not required.

The container preferably has means for controlling the temperature of the container and of the reactor located in the container. Heating and/or cooling elements permitting the temperature to be controlled are preferably placed/fixed in or on walls of the container.

The reactor or the container are mounted rotatably about a vertical axis during operation and are connected to a drive unit. The drive unit can set the reactor into motion in a rotating-oscillating manner about the positionally fixed, vertical axis, and therefore direct coupling of the drive unit to the reactor itself is not required. The forced coupling can also drastically reduce the release of electromagnetic beams which may, for example, cause sensor disturbances.

In order to realize the oscillating motion of the reactor, use is preferably made of an oscillating mechanism which is connected to the reactor or to the container via a suitable coupling means (for example via a toothed belt). The oscillating mechanism and reactor/container are preferably coupled below the reactor/container floor. Such an arrangement has the advantages of being very quiet and permitting a low center of gravity. The latter is relevant in particular in the case of large apparatus volumes and limited building height (for example for erection in laboratories).

The container can be inserted into a holder or an axial bearing from above, for example, with the aid of a crane, and therefore the same drive unit and/or the same measuring technology can be used for different types of container.

The reactor is preferably force-coupled to the drive unit in such a manner that the reactor rotation is accelerated and braked with a substantially constant angular acceleration or deceleration. As a result, in every phase of the rotating-oscillating reactor motion, instantaneous peak values of the hydrodynamic shearing forces on suspended particles (for example animal cells) are kept comparatively lower than in other forms of motion of the reactor. Gentle transitions between acceleration and braking up to the extreme case of a sinusoidal acceleration and deceleration profile are thoroughly desirable here in order to increase the operating duration of the drive elements. The permanent action of an acceleration or deceleration causes the rotational speed of the reactor to change in each motion phase of the rotating oscillation over time. Interconnected control modules are not required for this simple reactor motion.

As already mentioned, the container according to the invention has a floor which is curved inward into the interior space of the container and matched to the reactor according to the invention. Accordingly, as in the case of the reactor, the floor can be designed to be conical, in the manner of a spherical segment, to be bell-shaped, parabolic or in the shape of a polyhedron. The floor preferably has edges which are directed inward into the interior of the container. The floor is particularly preferably of pyramidal design.

The height h of the curvature of the container floor lies within the range of 0.01 times to 1 times the circle-equivalent diameter d of the floor cross section. If the floor cross section is, for example, square with a side length a, as may be the case in a pyramidally curved floor, the cross-sectional or area is A=a². The circle-equivalent diameter d is then, according to the formula 1:

$$d = \sqrt{\frac{4 A}{π}} \qquad ( 1 )$$

The height h of the curvature to the circle-equivalent diameter d is preferably within the range of 3% to 100%, particularly preferably within the range of 5% to 30% and very particularly preferably within the range of 10% to 20%.

By means of the inwardly curved floor and the preferably cuboidal embodiment of the container, distribution processes and/or mixing reactions can be carried out in combination with the rotating-oscillating motion in a simple manner and with the same intensity as in a conventional agitator container. By contrast, in the concept according to the invention, a shaft passage can be entirely omitted.

The inner curvature therefore firstly prevents the sedimentation of particles in the moving state because of an additional power input in the floor region. Secondly, it reduces the residual volume when removing the reactor contents via lateral connections above the floor edge of the reactor. Drainage connections reaching into the floor can thereby be avoided.

In addition, the stability of the container can be considerably increased, as already discussed above, by the concept of the inwardly curved container floor. As a result, the wall thicknesses of the lateral areas can be reduced and a weight and therefore cost saving achieved. Furthermore, by means of the reduction in the wall thicknesses, the forces of inertia thereof and ultimately the driving power for the container are considerably reduced.

In a particularly preferred embodiment, a connecting plate is preferably located in the floor region of the inner wall of the side channel, the connecting plate permitting the reactor to be supported in the region of the connecting lines and the connecting lines to be guided through into the side channel.

In a preferred embodiment, this connecting plate has passages for the passage of the connecting lines. Said connecting plate is connected to the reactor during assembly. In this embodiment, the connecting plate is preferably likewise a disposable element.

In a further preferred embodiment, the connecting plate is open laterally and is pushed laterally over the reactor connections in order to fix the deformable reactor in the container. In this case, the connecting plate can be reused and is part of the container.

The present invention furthermore relates to a device at least comprising a reactor according to the invention and a drive unit. If the reactor is designed in a deformable manner and therefore requires a container for supporting the reactor walls, the device according to the invention furthermore comprises a container according to the invention.

In a preferred embodiment, the device has a frame which surrounds the reactor (container) which rotates in an oscillating manner during operation. In a preferred embodiment, the frame has a door which, during operation of the reactor, is locked using a coded, counterfeit-proof key system. The latter is preferably used without the supply of current, while maintaining the maximum safety requirements regarding protection during working, and is therefore used without costly electronic monitoring by sensors in the frame and control system. The assembly of the frame is considerably simplified as a result. The frame has a door which permits operating staff access to the reactor only during a safe operational shutdown. The door and drive unit are protected by the locking system in such a manner that the reactor can only be put into operation when the door is closed. Use is preferably made for this purpose of a currentless securing system, also known to a person skilled in the art as a Fortis system. A single available dedicated installation key serves to lock the door and to control the drive unit. The drive unit can be started up only when the door is closed. When the key is removed from the control unit of the drive unit, the drive unit is switched off. The advantage of said coded single-key locking resides in inherent mechanical safety without electronic monitoring. It is not necessary to lay cables through the hollow pipes of the frame. This affords advantages in terms of cleaning, sealing, assembly and costs.

The present invention also relates to the use of a device comprising a reactor, optionally a container and a drive unit for cultivating cells and/or microorganisms.

In a preferred embodiment, a deformable reactor according to the invention is first of all placed into a container according to the invention which is coordinated with the reactor. The deformable reactor is preferably expanded by means of a gas and, in the semi-filled state, introduced into the container through the opening in the container. After the reactor has been positioned and locked in place by the opening, filling with medium takes place with the gas being displaced.

The reactor is preferably coupled via the connecting plate described to hose lines for, for example, supplying nutrient, and optionally a gas feed for the sparging, the drainage and to other couplings, for example for temperature sensors, pH sensors and the like. The gas removal from the head space and the gas feed for the surface gas introduction preferably take place at the reactor head.

In the disposable reactor, there is a ratio of liquid level to the reactor width of preferably 0.2 to 2 and particularly preferably 0.5 to 1. In addition, the reactor is operated, with the preferred hydrodynamic and process engineering properties thereof being maintained, with a sufficient head space between the reactor head and liquid level of at least 5% to 50% of the height of the liquid, and preferably at least 25% of the height of the liquid.

The drive unit coupled to the reactor or container ensures, by means of a rotating-oscillating motion, thorough mixing of the reactor contents.

It has surprisingly turned out that a comparatively small angle amplitude for the rotating-oscillating motion of the reactor suffices in order to achieve thorough mixing and/or sufficient intensification of transport processes. In particular, it is hardly required to realize 3600° revolutions (which corresponds to 10 revolutions) of the reactor, and therefore structurally complex solutions for coupling the oscillating-rotating reactor to the nonmoving surroundings (for example for feeding in and removing media and gases, electric power and electric signals) are not required.

In the use according to the invention, the reactor is moved in a rotating-oscillating manner with an angle amplitude α in the region of 2°≦|α|≦3600°, preferably 20°≦|α|≦180°, particularly preferably 45°≦|α|≦90°, wherein deviations of ±5° may be present. In particular, |α|=60° is very particularly preferred in the use of particularly low-shear bioreactors with surface gas introduction. In total, the oscillating motion therefore passes through an angle of 2|α|.

Tests have shown that, when the power input is increased, motion states can be set in the reactor, in which gas bubbles are introduced into the reactor medium. For cells and/or microorganisms which are not damaged by sparging, a very simple supply of gas can thereby be realized.

It has been shown that an undesirable production of foam initially rises, as anticipated, as the reactor motion increases in order then, however, after passing through a maximum foam height, to drop again to readily controllable foam heights of a few centimeters. The cause of said highly astonishing phenomenon of said foam destruction resides in the fact that, in said motion states of the liquid, not only the gas located in the head space but also the foam itself is sucked in from the surface. The foam is dissolved again gently, i.e. with strict avoidance of bursting the gas bubbles, by being sucked in again under the surface of the liquid without shearing forces being applied. In particular, it is possible to produce a shaft flow, by means of which some of the reactor contents on the surface are conveyed into the interior of the reactor contents. In this preferred type of reactor, formation of foam can therefore be substantially suppressed and at the same time a particularly gentle and effective surface gas introduction can be realized. However, the use of the oscillating foam destroyer is in no way limited to reactors with surface gas introduction but can advantageously be used in general in sparged reactors.

As the reactor volume increases, it becomes increasingly difficult to sufficiently supply and remove cells and/or microorganisms with/from gaseous substances via surface gas introduction. In a particularly preferred embodiment, gas is therefore introduced to the reactor via one or more gas distributors fixed laterally in the floor region. If, for the sake of simplicity, a punctiform gas feed takes place via a single gas distributor, gas bubbles rise in the form of zig-zag-shaped lines as a consequence of the rotating-oscillating motion of the reactor and of the reactor medium motion which lags behind the external movement of the reactor. Therefore, even with puntiform introduction, by means of the rotating-oscillating motion the gas bubbles are distributed along the wall in which the gas distributor is fixed. At the same time, as a consequence of the difference in density between the reactor medium region in which gas bubbles rise and the region which is remote from the gas-distributor point, a circular flow ensures that the reactor contents are circulated.

The mixing action of the rotating-oscillating motion is further supported by the angular shape of the reactor/container, on the one hand, and by the inwardly curved floor, on the other hand.

One or more gas distributors are placed into the disposable reactor in the floor region preferably via the connecting plate. A gas distributer can be designed for introduction of coarse gas bubbles as an opening with a diameter of 0.5 mm to 10 mm or as a short perforated hose or pipe section with an average pore diameter of 0.5 mm to 1 mm. Use is preferably made of introduction of fine gas bubbles, for example by means of a sintering candle with sintering grain sizes of 0.2 μm to 50 μm. Gas introduction devices can be designed as short pipe sections at right angles to the container wall or as longer pipe sections parallel to the container wall, wherein said pipe sections have to be supplied with gas at one point and, depending on length, have to be secured at a further point.

The invention is explained in more detail below with reference to figures without, however, restricting said invention to the embodiments shown.

In the figures:

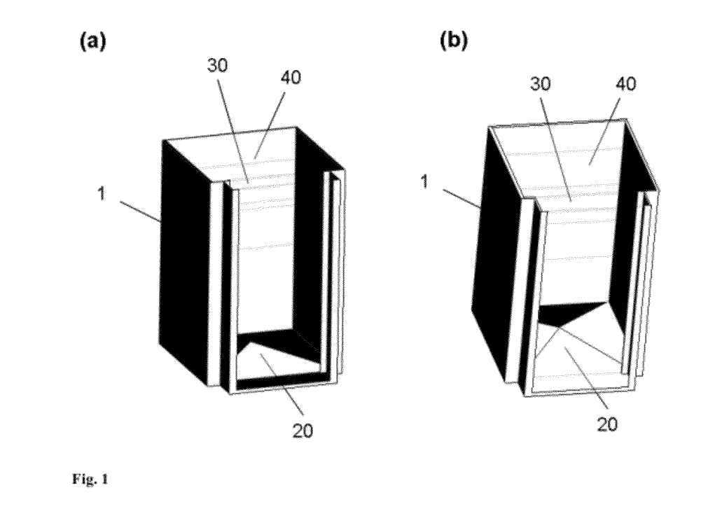

FIGS. 1(a),(b) show a perspective illustration of the container according to the invention

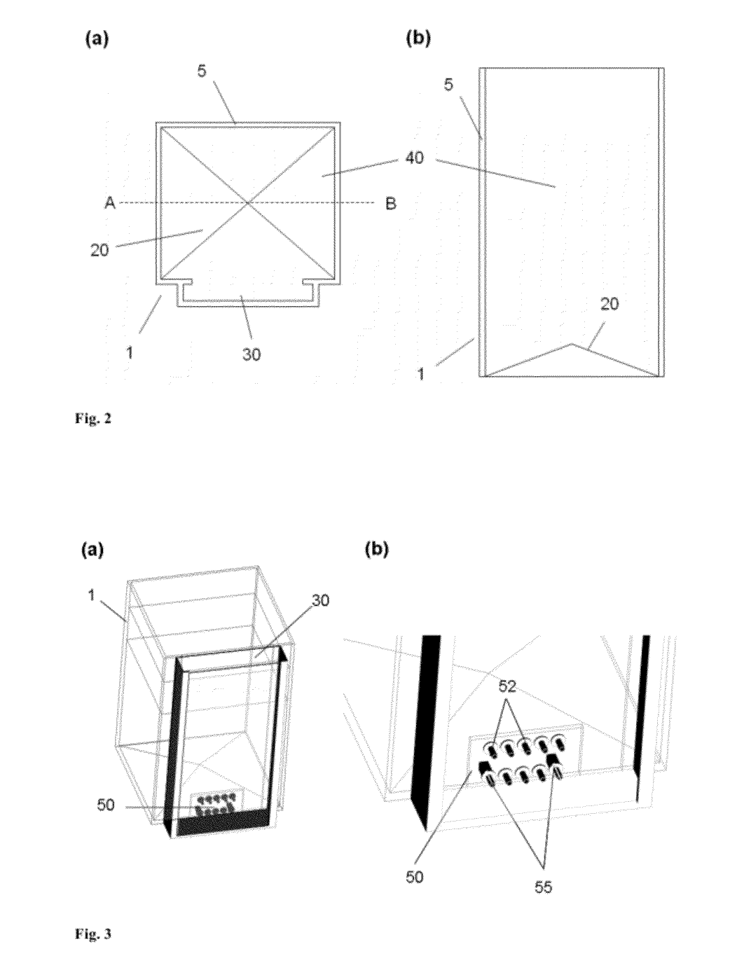

FIG. 2(a) shows a schematic illustration of the container according to the invention in a cross section from above

FIG. 2(b) shows a schematic illustration of the container according to the invention in a cross section through the line shown in FIG. 2(a) between the points A and B

FIGS. 3(a),(b) show a perspective illustration of the container according to the invention with a connecting plate

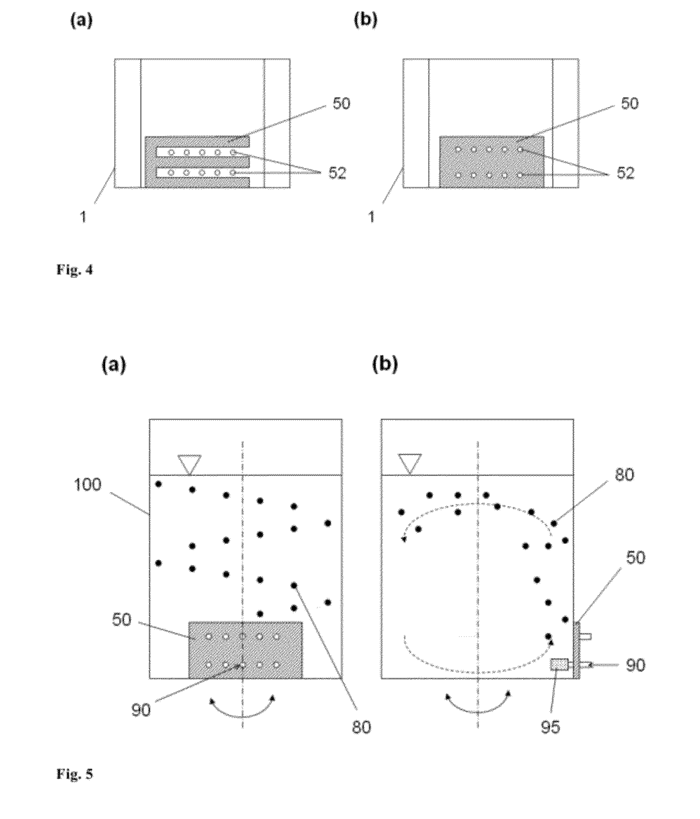

FIGS. 4(a),(b) show various embodiments of a connecting plate

FIGS. 5(a),(b) show a schematic illustration of the distribution of gas bubbles in a rotating-oscillating disposable reactor

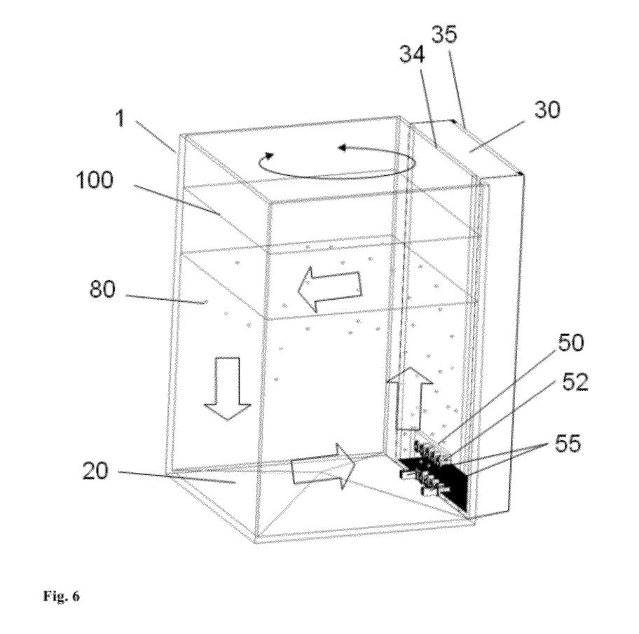

FIG. 6 shows a perspective illustration of a combination of container and disposable reactor with gas introduced

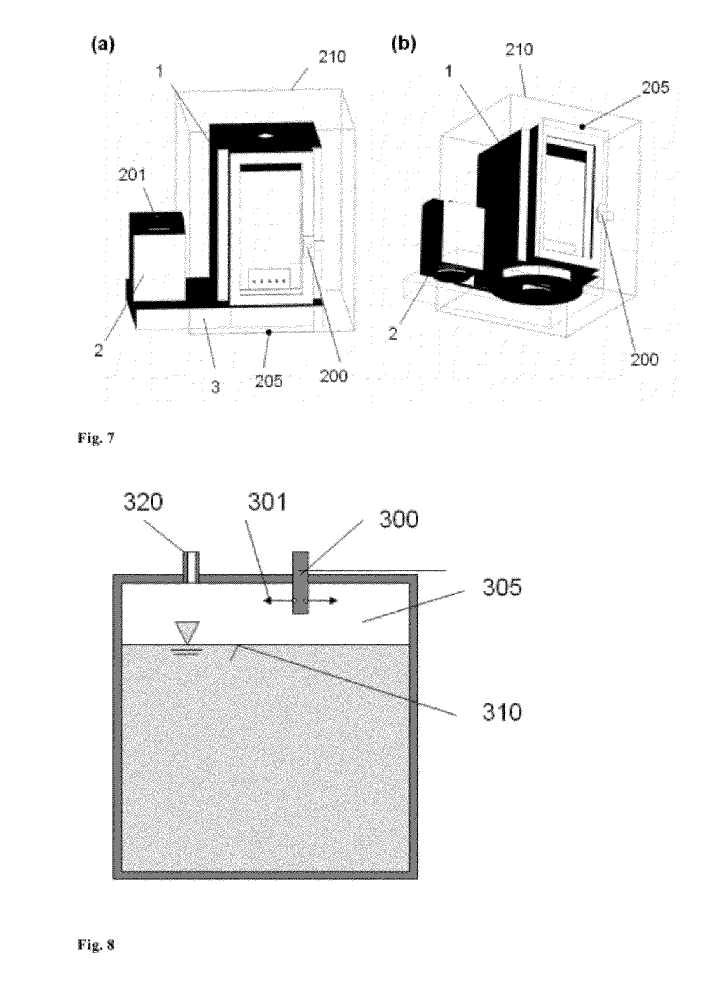

FIGS. 7(a),(b) show a perspective illustration of a combination of container and drive unit

FIG. 8 shows a schematic illustration of a container and reactor with surface gas introduction

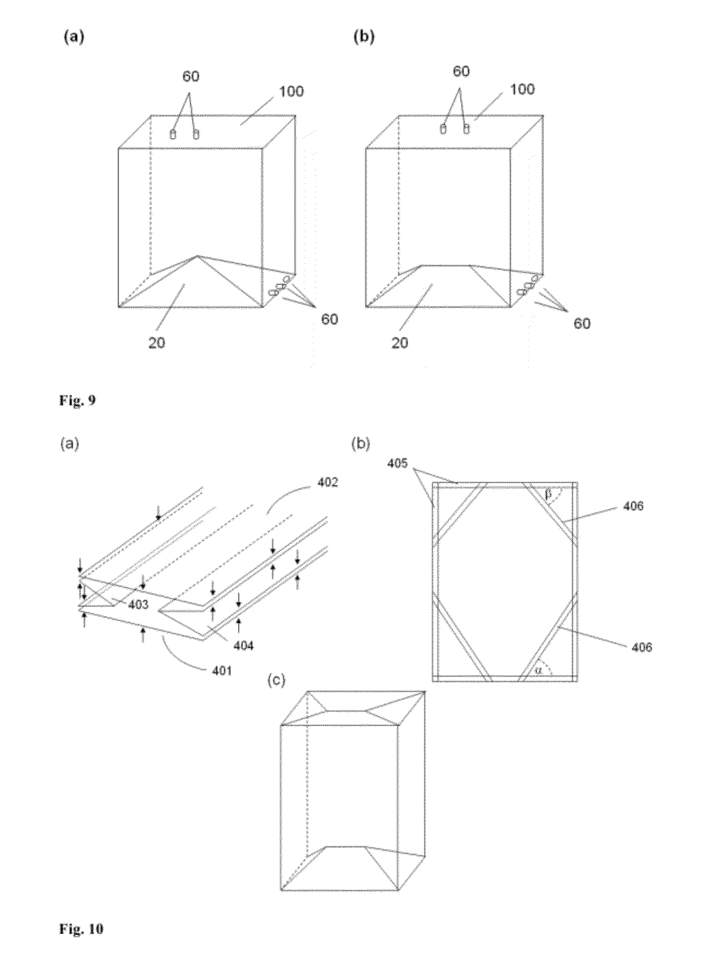

FIGS. 9(a),(b) show a schematic illustration of reactors according to the invention

FIGS. 10(a),(b),(c) show a schematic illustration of a method for producing a reactor according to the invention.

FIG. 1 shows schematically a preferred embodiment of the container 1 according to the invention in a perspective illustration from the front (a) and obliquely from above (b). The container has a floor 20 curved pyramidally into the interior space 40. The container has an interior space 40 for accommodating a reactor, and a side channel 30 for the guiding of connecting lines.

FIG. 2(a) shows the container 1 from FIG. 1 in a cross section from above. FIG. 2(b) shows the container 1 in a cross section along the dashed line, shown in FIG. 2(a), between the points A and B.

FIG. 3(a) shows a preferred embodiment of the container 1 according to the invention in a perspective illustration. A connecting plate 50 is fixed in the floor region of the container. That region of the container 1 that comprises the connecting plate 50 is illustrated in an enlarged scale in FIG. 3(b). In the embodiment shown, the connecting plate 50 comprises connecting points 52 which are joined on one side to a reactor. On the other side facing away from the reactor, it is possible to fit, for example, hose lines for supplying media. The connecting plate may also have connections for probes (for example a pH probe, temperature probe). In the embodiment shown, gas distributors 55 are integrated in the connecting plate.

FIGS. 4(a) and (b) show various embodiments of a connecting plate 50 fixed in the floor region of a container 1. The connecting plate 50 is open laterally in FIG. 4(a) and is pushed laterally over the connections 52 of the reactor in order to fix the reactor in the container. The connecting plate can be reused and is part of the container. The connecting plate 50 in FIG. 4(b) has passages for the guidance of the connecting lines. It is connected to the reactor during assembly. In this embodiment, the connecting plate itself is also a disposable element.

FIGS. 5(a) and 5(b) show the introduction of gas to a reactor via a connecting plate 50 in a schematic illustration. In FIG. 5(a), the connecting plate is directed toward the observer and in FIG. 5(b) the connecting plate 50 shown from the side. Gas 90 is guided into the reactor in a punctiform manner via a connection. Use is preferably made here of a short sintering candle 95 in order to ensure the introduction of fine gas bubbles. In order to produce small gas bubbles, the rotating-oscillating motion (indicated by the double arrow) prevents bubble coalescence on the surface of the gas introduction device as a result of the latter being approached by the inert fluid, and causes bubbles to be dispensed in a zig-zag-shaped manner, i.e. the gas bubbles 80 rise in the form of a zig-zag line and are therefore distributed over the entire width of the reactor. The introduction of gas via the reactor wall also results in a circulating flow (illustrated by the dashed arrows).

FIG. 6 shows the introduction of gas to the reactor 100 in an oscillating container 1 in a perspective illustration. In this embodiment, gas is placed into the reactor via two gas distributors 55. The oscillating-rotating motion (illustrated by the double arrow) distributes the gas bubbles 80. The circulating flow (illustrated by the thick arrows) ensures that the gas bubbles pass even into the reactor regions which are located remotely from the gas entry points into the reactor. In the case of cultivating cells or microorganisms, the circulating flow ensures that the cells or microorganisms are held in suspended form in the reactor. By means of the inwardly curved floor 20, which is of pyramidal design in the present example, vortices are produced which swirl up and re-suspend dropping cells or microorganisms.

FIG. 6 also shows a preferred embodiment of the side channel 30. It runs between the two spaced-apart walls 34, 35, which are preferably of transparent design, into the floor region of the reactor. The inner wall 34 which is directed toward the reactor serves to support the preferably deformable reactor. The outer wall 35 serves to seal off the container to the outside. Both walls can be removed for the frontal introduction of the reactor into the container.

FIG. 7 shows by way of example a container 1 according to the invention which is fixed together with a drive unit 2 on a common floor plate 3. The container 1 is fixed on the floor plate 3 in a manner mounted rotatably with respect to a vertical axis. In the present example, the drive unit comprises an oscillating mechanism which is connected via a toothed belt to the container and can set the latter into a rotating-oscillating motion.

In a preferred embodiment, the rotating container is surrounded by a frame 210 which, when used correctly, reliably prevents access to operating staff during operation. The frame 210 has at least one door 205 which permits operating staff access to the container and reactor only when an operational shutdown is ensured. Doors 205 and drive unit are protected by a suitable locking system in such a manner that the container can only be started up when the doors 205 are closed. Use is preferably made for this purpose of a currentless securing system which is also known to a person skilled in the art as a Fortis system. A single available dedicated installation key, when removed from the switching housing 201, switches off the current feed to the drive unit before said key can be used for locking the locking device 200 which can only be actuated in the closed state of the door 205. The advantage of the coded single key locking resides in inherent mechanical safety without electronic monitoring. It is not necessary to lay cables through the hollow pipes of the frame. This affords advantages in terms of cleaning, sealing, assembly and costs.

FIG. 8 shows by way of example the gas feed into a reactor with surface gas introduction. In order to prevent a short-circuiting flow between the gas feed 300 and gas removal 320, the gas flow is deflected in the horizontal direction by means of one or more gas distributors and is accelerated through corresponding openings to higher speeds with pressure losses being accepted. Preferred pressure losses lie between 1 mbar and 5000 mbar. The pressure losses particularly preferably lie between 10 mbar and 500 mbar. By means of the horizontal gas jets 301 produced in this manner, a simple, cost-effective and efficient mixing of all of the gas is made possible in the head space 305 of the reactor.

FIG. 9 shows schematically two polyhedral exemplary embodiments of a reactor 100 according to the invention. The reactor comprises a space which is limited by preferably flexibly designed (deformable) walls. The floor 20 projects into the interior of the space. FIG. 9(a) shows a floor which is fitted/curved inward pyramidally and tapers to a point. In FIG. 9(b), the upper region of the pyramid is designed as an edge.

Passages and/or terminations 60 are fixed in the walls in order to provide coupling between the interior space of the reactor and the outside world.

FIG. 10 shows schematically how a reactor according to the invention made of polymer films can be produced analogously to the manner described in U.S. Pat. No. 6,186,932B1. In the example, the reactor is joined together from four film parts (401, 402, 403, 404). For this purpose, the films are placed together in the manner shown in FIG. 5(a): the film part 401 lies lowermost, the film part 402 lies uppermost, and the film parts 403 and 404 are placed in folded form between the film parts 401 and 402. The film parts are welded together around the borders (indicated by the arrows), thus producing a closed bag. FIG. 5(b) shows the film arrangement in FIG. 5(a) from above. Welded seams 405 can be seen around the film arrangement. In addition, the film assembly has welded seams 406 which extend over the corners. The floor and the head shape of the reactor, in the case of the floor in particular the size of the curvature of the floor, are determined by the sizes of the angles α and β.

The corners of the film arrangement from FIG. 5(b) are trimmed to size after the corner welded seams 406 are produced.

### REFERENCE NUMBERS

1 Container
2 Drive unit
3 Floor plate
5 Container wall
20 Inwardly curved floor
30 Side channel
34 Channel wall
35 Channel wall
40 Interior space
50 Connecting plate
52 Connections
55 Gas distributors
60 Passages/connections
80 Gas bubbles
90 Gas feed
100 Reactor
200 Closing device
201 Control unit
205 Door
210 Frame
300 Gas feed
301 Gas jet
305 Head space
310 Liquid level
320 Gas removal
401 Film piece
402 Film piece
403 Film piece
404 Film piece
405 Welded seam on the longitudinal side
406 Welded seam over corner

1. A reactor comprising walls, enclosing an interior space, and passages and/or connections in the walls, permitting connection of the interior space to the external surroundings, wherein the reactor, when filled with liquid, has an angular cross section parallel to the surface of the liquid and a floor which is curved or curvable into the interior space.

2. The reactor according to claim 1, wherein the floor, curved or curvable inward, has edges projecting into the interior space of the reactor.

3. The reactor according to claim 1, wherein the floor, which is curved or curvable inward, is of polyhedral design.

4. The reactor according to claim 1, wherein a height h of a curvature of the floor in a ratio to a circle-equivalent diameter d of the floor is within a range of 3% to 100%.

5. The reactor according to claim 1, wherein the passages and/or connections are provided in a side region of the reactor located adjacent to an edge of the floor.

6. The reactor according to claim 1, wherein the walls are flexible and therefore the reactor is adapted to be deformable.

7. A container for accommodating the reactor according to claim 1, the container at least comprising
an interior space for accommodating the reactor,
an opening for frontal introduction of the reactor into the container, wherein the opening is closable and is adapted to be liquid-tight in a closed state,
a floor which is curved inward into the interior space of the container, and
a side channel, via which hoses and channels can be led up to the reactor.

8. The container according to claim 7, wherein the inwardly curved floor is of polyhedral design.

9. The container according to claim 7, wherein the side channel runs between two spaced-apart walls, the walls reaching into a floor region of the reactor, and the walls being removable for the frontal introduction of the reactor into the container.

10. The container according to claim 7, further comprising a connecting plate, arranged laterally in a region of the curved floor, for the fixing and/or guiding of couplings and/or hoses which are connectable to the reactor.

11. The container according to claim 10, wherein the connecting plate is open laterally and, in order to fix the reactor in the container, is pushable laterally over the connections of the reactor.

12. The container according to claim 7, further comprising heating and/or cooling elements mounted in and/or on walls of the container.

13. A device at least comprising the reactor according to claim 1, a drive unit for generating a rotating-oscillating motion of the reactor, and, if the reactor is adapted to be deformable, optionally a container at least comprising
an interior space for accommodating the reactor,
an opening for frontal introduction of the reactor into the container, wherein the opening is closable and is adapted to be liquid-tight in a closed state,
a floor which is curved inward into the interior space of the container, and
a side channel, via which hoses and channels can be led up to the reactor.

14. The device according to claim 13, further comprising a frame and a locking system permitting access to the reactor only during an operational shutdown.

15. A method comprising:
cultivating cells and/or microorganisms within the reactor of the device according to claim 13.

16. The reactor according to claim 1, wherein the floor, curved or curvable inward, is of pyramidal design.

17. The reactor according to claim 4, wherein the height h of the curvature of the floor in the ratio to the circle-equivalent diameter d of the floor is within a range of 5% to 30%.

18. The reactor according to claim 17, wherein the height h of the curvature of the floor in the ratio to the circle-equivalent diameter d of the floor is within a range of 10% to 20%.

19. The container according to claim 7, wherein the inwardly curved floor is of pyramidal design.
